SEQUENCE LISTING (9 sequences)

SEQ ID NO: 1 - DNA, 1761 bp, Artificial Sequence
  Recombinant gene
atggccaagacaattgcgtacgacgaagaggcccgtcgcggcctcgagcggggcttgaacgccctcgccgatgcggtaaaggtgacattgggccccaagggccgcaacgtcgtcctggaaaagaagtggggtgcccccacgatcaccaacgatggtgtgtccatcgccaaggagatcgagctggaggatccgtacgagaagatcggcgccgagctggtcaaagaggtagccaagaagaccgatgacgtcgccggtgacggcaccacgacggccaccgtgctggcccaggcgttggttcgcgagggcctgcgcaacgtcgcggccggcgccaacccgctcggtctcaaacgcggcatcgaaaaggccgtggagaaggtcaccgagaccctgctcaagggcgccaaggaggtcgagaccaaggagcagattgcggccaccgcagcgatttcggcgggtgaccagtccatcggtgacctgatcgccgaggcgatggacaaggtgggcaacgagggcgtcatcaccgtcgaggagtccaacacctttgggctgcagctcgagctcaccgagggtatgcggttcgacaagggctacatctcggggtacttcgtgaccgacccggagcgtcaggaggcggtcctggaggacccctacatcctgctggtcagctccaaggtgtccactgtcaaggatctgctgccgctgctcgagaaggtcatcggagccggtaagccgctgctgatcatcgccgaggacgtcgagggcgaggcgctgtccaccctggtcgtcaacaagatccgcggcaccttcaagtcggtggcggtcaaggctcccggcttcggcgaccgccgcaaggcgatgctgcaggatatggccattctcaccggtggtcaggtgatcagcgaagaggtcggcctgacgctggagaacgccgacctgtcgctgctaggcaaggcccgcaaggtcgtggtcaccaaggacgagaccaccatcgtcgagggcgccggtgacaccgacgccatcgccggacgagtggcccagatccgccaggagatcgagaacagcgactccgactacgaccgtgagaagctgcaggagcggctggccaagctggccggtggtgtcgcggtgatcaaggccggtgccgccaccgaggtcgaactcaaggagcgcaagcaccgcatcgaggatgcggttcgcaatgccaaggccgccgtcgaggagggcatcgtcgccggtgggggtgtgacgctgttgcaagcggccccgaccctggacgagctgaagctcgaaggcgacgaggcgaccggcgccaacatcgtgaaggtggcgctggaggccccgctgaagcagatcgccttcaactccgggctggagccgggcgtggtggccgagaaggtgcgcaacctgccggctggccacggactgaacgctcagaccggtgtctacgaggatctgctcgctgccggcgttgctgacccggtcaaggtgacccgttcggcgctgcagaatgcggcgtccatcgcggggctgttcctgaccaccgaggccgtcgttgccgacaagccggaaaaggagaaggcttccgttcccggtggcggcgacatgggtggcatggatttccatatggctagcgaattcggttctaccgctccgccggctcacggtgttacctctgctccggacacccgtccggctccgggttctaccgctccgccggctcacggtgttacctctgctccggacaaccgtccggctctgtga

SEQ ID NO: 2 - protein, 586 aa, Artificial Sequence
  Recombinant gene
MAKTIAYDEEARRGLERGLNALADAVKVTLGPKGRNVVLEKKWGAPTITNDGVSIAKEIELEDPYEKIGAELVKEVAKKTDDVAGDGTTTATVLAQALVREGLRNVAAGANPLGLKRGIEKAVEKVTETLLKGAKEVETKEQIAATAAISAGDQSIGDLIAEAMDKVGNEGVITVEESNTFGLQLELTEGMRFDKGYISGYFVTDPERQEAVLEDPYILLVSSKVSTVKDLLPLLEKVIGAGKPLLIIAEDVEGEALSTLVVNKIRGTFKSVAVKAPGFGDRRKAMLQDMAILTGGQVISEEVGLTLENADLSLLGKARKVVVTKDETTIVEGAGDTDAIAGRVAQIRQEIENSDSDYDREKLQERLAKLAGGVAVIKAGAATEVELKERKHRIEDAVRNAKAAVEEGIVAGGGVTLLQAAPTLDELKLEGDEATGANIVKVALEAPLKQIAFNSGLEPGVVAEKVRNLPAGHGLNAQTGVYEDLLAAGVADPVKVTRSALQNAASIAGLFLTTEAVVADKPEKEKASVPGGGDMGGMDFHMASEFGSTAPPAHGVTSAPDTRPAPGSTAPPAHGVTSAPDNRPAL

SEQ ID NO: 3 - DNA, 120 bp, Homo sapiens
  CDS (1)..(120)
ggttctaccgctccgccggctcacggtgttacctctgctccggacacccgtccggctccgggttctaccgctccgccggctcacggtgttacctctgctccggacaaccgtccggctctg

SEQ ID NO: 4 - protein, 40 aa, Homo sapiens
GSTAPPAHGVTSAPDTRPAPGSTAPPAHGVTSAPDNRPAL

SEQ ID NO: 5 - DNA, 20 bp, Artificial Sequence
  Primer
ccatggccaagacaattgcg

SEQ ID NO: 6 - DNA, 37 bp, Artificial Sequence
  Primer
cgaattcgctagccatatggaaatccatgccacccat

SEQ ID NO: 7 - DNA, 24 bp, Artificial Sequence
  Primer
ttcgccatggccaagacaattgcg

SEQ ID NO: 8 - DNA, 162 bp, Artificial Sequence
  Primer
ggccgcaagcttttatcacagagccggacggttgtccggagcagaggtaacaccgtgagccggcggagcggtagaacccggagccggacgggtgtccggagcagaggtaacaccgtgagccggcggagcggtagaaccgaattcgctagccatatgcaaatc

SEQ ID NO: 9 - protein, 159 aa, Artificial Sequence
  Fusion Protein
MRRRRRRRREFRSGSTAPPAHGVTSAPDTRPAPGSTAPPAHGVTSAPDNRPALKLAAGSGSTAPPAHGVTSAPDTRPAPGSTAPPAHGVTSAPDNRPALKLAAGSGSTAPPAHGVTSAPDTRPAPGSTAPPAHGVTSAPDNRPALKLAAGSLEHHHHHH

(19) United States
(12) Patent Application Publication
(10) Pub. No.: US 2005/0031649 A1
(43) Pub. Date: Feb. 10, 2005

(54) Recombinant fusion proteins comprising BCG heat shock protein 65 and the epitope of MUC1

(76) Inventors: Yongli Yu, Changchun (CN); Hui Li, Changchun (CN); Liying Wang, Changchun (CN)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 10/635,211

(22) Filed: Aug. 6, 2003

(51) Int. Cl. C07H 21/04; C12P 21/04; A61K 39/07; C12N 1/21

(52) U.S. Cl. 424/246.1; 435/69.7; 435/252.31; 435/320.1; 530/350; 536/23.7

(57) ABSTRACT

Certain embodiments described herein provide a fusion protein comprising Bacillus Calmette Guérin (BCG) heat shock protein 65 (HSP65) and 1 to 5 copies of the epitope of MUC1, which is therapeutic and/or preventive to human MUC-1 expressing carcinomas including breast cancer, ovarian cancer, colon cancer, prostate cancer and lung cancer. Certain embodiments described herein provide a nucleic acid encoding the fusion protein, a recombinant plasmid containing the nucleic acid, a host cell transformed with the recombinant plasmid and/or a method for preparing the fusion protein by culturing the host cells.

### CROSS REFERENCE TO RELATED APPLICATIONS

The present application is related to commonly owned and assigned U.S. patent application Ser. No. ______, filed Aug. 6, 2003, entitled “Recombinant Fusion Proteins Comprising BCG Heat Shock Protein 65 and the Epitope of Human Prostate Specific Antigen”, which is hereby incorporated by reference herein.

### FIELD OF THE INVENTION

The present invention generally relates to a fusion protein effective to treat and/or prevent human carcinomas. More particularly, the present invention relates to a recombinant fusion protein comprising heat shock protein 65 (hereinafter, it is also referred to as “HSP65”) fused to the epitope of MUC1 (hereinafter, it is also referred to as MUC1-ME), especially a recombinant fusion protein HSP65-MUC1 effective to treat and/or prevent human MUC-1 expressing carcinomas. The present invention also relates to the nucleic acid encoding the recombinant fusion protein HSP65-MUC1, a recombinant vector or plasmid containing the nucleic acid, a host cell transformed with the recombinant plasmid and a method for preparing the fusion protein by culturing the transformed host cell.

### BACKGROUND OF THE INVENTION

Mucins belong to a protein family comprising a group of proteins present on the surface of epithelial cells of various organs (Segal-Eiras A, et al. Breast cancer associated mucin: a review. Allergol Immuopathol (Madr) 1997 Jul.-Aug. , 25). At present, 14 mucins have been identified, including MUC1, MUC2, MUC3 (3A 3B), MUC4, MUC5A,C; MUC5B, MUC6, MUC7, MUC8, MUC9, MUC10, MUC11, MUC12 and MUC13

Human MUC1 gene locates on chromosome 1(1q21) and comprises 7 extrons. The extron 1 encodes a signal peptide and extron 2 encodes variable number of tandem repeats (VNTRs) of the MUC1 protein. The VNTRs form the polymorphism of MUC 1 gene. The MUC 1 alleles with different numbers of VNTRs exist in human population. Each individual has different numbers of VNTRs from each other, ranging from 20-120 (Gendler S. J, Cancer Genet Cytogenet. 1998 January 1;100(1):63-7). In human, the two most common MUC1 alleles have 41 and 85 VNTRs, respectively.

Full length of human MUC1 cDNAs are obtained from cDNA libraries of human T lymphocytes, breast epithelial cells, pancreatic cancer cell and ovarian cancer cells.

A small amount of MUC1 protein is expressed on the surface of normal cells in breast, ovary, placenta, urinogenital duct, saliva gland, oral cavity, gastrointestinal epithelium, hematopoietic cells, activated T lymphocytes, B cells and dendritic cells. MUC1 protein can be over-expressed in the cells of breast cancer, ovarian cancer, pancreatic cancer, prostate cancer, bladder cancer, melanoma and laryngeal cancers. The transcription and translation of MUC 1 genes are enhanced in the malignant cells.

High level of MUC1 proteins can be detected in the blood of some of breast cancer and ovarian cancer patients (M. R. Price. Eur. J. Cancer Clin. Oncol. 24 1988, pp. 1799-1804. M. A. Reddish, et al. Cancer Immunol. Immunother. 42. 1966, pp. 303-309; H. Kobayashi, et al. J. Clin. Oncol. 10,1992, pp. 95-101) and can be used to diagnose the recurrence of breast cancer.

MUC1 protein synthesized in the cells can be processed, through a MHC class I pathway, into small peptides (may be epitopes) which will be presented, associated with MHC class I molecules, on the surface of the cells and recognized by specific cytotoxic T lymphocytes (CTLs) (Chan AK et al, Int J Cancer. 1999 August 27;82(5):721-6). Evidence that MUC1-specific antibodies and MHC-restricted MUC1-specific CTLs are present in patients of breast cancer, ovarian cancer, pancreatic cancer demonstrates that MUC1 can elicit anti-tumor cellular immune response targeting at MUC1 (Snijdewint FG, Cancer Immunol Immunother. 1999 April;48(l):47-55).

Breast cancer cells derived from 91 percent of breast cancer patients express high levels of MUC1, determined by MUC-1 specific antibody staining. In patients with kidney cancer, augmentation of MUC1 expression is associated with enlargement of tumor dimension, metastasis and invasion into big veins (Kraus S, Hum Pathol. 2002 January;33(1):60-7). High level expression of MUC1 in the patients of stomach cancer, liver cancer, colon cancer, lung cancer, head and neck squamous carcinoma and cervical cancer imply a poor prognosis (Lee H. S et al. Cancer. 2001 September 15;92(6):1427-34. Matsumura N et al. Cancer. 2002 March 15;94(6):1770-6; Jang KT et al. J Korean Med Sci. 2002 February;17(1):29-33. ; Li A, et al. Pathol Int. 2001 November;51(11):853-60). High level expression of MUC-1 was also detected in 50.9% of thyroid cancer, 67% of acute myelogenous leukemia, 92% of osteoma (Brugger W et al, Cancer Res. 2001 September 15;61(18):6846-50), 75% of esophageal squamous cell carcinoma (Flucke U. et al, Anticancer Res. 2001 May-June;21(3C):2189-93) and 50-95% of lymphoma patients. No MUC1 expression was found in noninvasive pancreatic mucilaginous adenocarcinoma. When it developed into invasive adenocarcinoma, MUC1 was expressed, which indicated that MUC1 expression level is related to tumor metastasis. Since high level expression of MUC1 is associated with tumor invasion and metastasis, MUC1 can be used as label molecule indicating malignant transformation of normal cells and producing potent protective cellular immune response of the body (Croce MV et al, Pathol Oncol Res. 2001;7(4):284-91).

MUC1 expression can be increased by 100 times in tumor cells, with a large amount of MUC1 polypeptides binding to MHC class I molecules. The epitopes of the MUC1 protein are localized in the tandem repeat domain, which can elicit mice to produce specific antibodies (I. F. C. Mckenzie and P.-X. Xing, Mucins in breast cancer-recent advances. Cancer Cells 2(1990), pp. 75-78).

All of this evidence indicates that MUC1 expression is closely associated with a variety of cancers. One approach to using MUC1 as a basis for treatment of cancer is described by Michelle D. Winthrop in University of California Medical Center, who developed a MUC1-specific double-function antibody. The antibody can not only recognize MUC1 but also bind to radiating chelate ⁹⁰Y-DOTA. A single chain antibody against MUC1 (scFv,30kD) can bind effectively to breast cancer cell expressing MUC1, and then capture an applied cytotoxic isotope (Winthrop MD, et al. Antibody phage display applications for nuclear medicine imaging and therapy. Q J Nul Med. 2000, 44(3):284-95).

Other evidence, however, suggests that an immune response to MUC1, especially a cellular immune response, reduces or controls some cancers. For example, in mice with spontaneous pancreatic cancer, MUC1-specific CTLs are stimulated. Transferring the CTLs into the mice can inhibit the growth of MUC1 positive tumor (Mukherjee P et al, J Immunol. 2000 September 15;165(6):3451-60). In C57/BL6 mice immunized with the MUC1 gene-carrying plasmid (pCI-MUC1), the growth of the tumor was inhibited (Johnen H et al, Cancer Immunol Immunother. 2001 September;50(7):356-60).

Mice immunized with oxidized mannan-MUC1 as a fusion protein generated highly active CD8⁺ CTLs to MUC1 peptides. Tumor formation was prevented and tumor dimension was reduced (Apostolopoulos V et al, Vaccine. 1996 June;14(9):930-8). Apostolopoulos V et al used mannan-MUC1 peptide to immunize animals, inducing specific CTL which provide protection against tumor challenge (Apostolopoulos V, et al. Cyclosphosphamide enhances the CTL precursor frequency in mice immunized with MUC1 -mannan fusion protein(M-FP), J Immunotherapy, 1998 March; 21(2): 109-13).

Keyhole limpet hemocyanin-MUC1 fusion protein can induce specific CTLs, and the CTLs can kill MCF7 cells which express MUC1 (Gilewski T et al, Cancer Res. 2000 May;6(5):1693-701).

Mice immunized with human MUC1 generated H-2 restricted CD8⁺ CTLs (V Apostolopoulos, CTL in mice immunized with human MUC1 are MHC-restricted. J. Immunol. 1155(1995),pp. 5089-5094). HLA-A0201-binding peptides are in MUC1 VNTRs, and MUC1 can induce HLA-A2 restricted CTLs to MUC1 human breast cancer (V Apostolopoulos, Induction of HLA-A2-restricted CTLs to mucinl human breast cancer antigen. J. Immunol. 159(1997), pp5211-5218).

Mice immunized with mannan-mucinl as a fusion protein induced MHC class I restricted CTL, and the CTL can kill breast cancer cells (Lee CJ, et al. The effect of T1 and T2 cytokines on the cytotoxic T cell response to mannan-MUC1. Cancer Immunol Immunother 2000 February; 48(11): 644-52).

Breast cancer cell-specific CTL can be induced in vitro with human dendritic cells pulsed with MUC1 CTL epitope peptides (Brossart P, et al. Identification of HLA-A2-restricted T cell epitopes derived from the MUC1 tumor antigen for broadly applicable vaccine therapies. Blood 1999 June 15; 93(12):4309-17).

Sixteen metastatic breast cancer patients were immunized with a sixteen amino acid MUC1 peptides conjugated to KLH, seven of them generated MHC class I-restricted CTL which can kill tumor cells expressing MUC1 (Reddish M; et al. Anti-muc1 class I restricted CTLs in metastatic breast cancer patients immunized with a synthetic MUC1 peptide, Int J Cancer 1998 June 10; 76(6):817-23). Twenty five patients with carcinoma of colon, stomach and rectum were immunized with MUC1-mannan fusion protein, T cell proliferation was found in 2 out of 25 patients (Karanikas V, et al. Antibody and T cell responses of patients with adenocarcinoma immunized with mannan-MUC1 fusion protein. J Clin Invest 1997 December 1;100(11):2783-92).

Goydos J S et al vaccinated 63 patients with breast cancer with a 15 aa mucin peptide mixed with BCG, and observed that MUC1-specific CTLs were induced (Goydos JS, et al. A phase I trial of a synthetic mucin peptide vaccine. Induction of specific immune reactivity in patients with adenocarcinoma).

All of these experiments have shown that the creation of an immune response to MUC1 could be an effective treatment for cancer.

### DESCRIPTION OF THE INVENTION

Materials and methods for creating an immune response to MUC1 are described herein, including embodiments for therapeutic, prophylactic, diagnostic, and research-tool purposes. An aspect of the invention is a molecule that has an element of MUC1 but also has an element that elicits an immune response in a patient such that the patient's immune system reacts to MUC1. A preferred molecule is a recombinant fusion protein that has a portion that is similar to at least a portion of MUC1 and also has a portion that comprises at least a portion of Bacillus Calmette Guérin heat shock protein 65. Such a fusion protein will be useful for treating cancer, for reasons discussed below.

Cytotoxic T lymphocytes (CTL) are the most effective tumor killing cells in immune system. Therefore, treatments that elicit an immune response will preferably elicit responses mediated by CTLs. To stimulate anti-tumor immunity, therefore, a recombinant protein should be able to generate tumor specific CTLs. Usually, upon immunization, exogenously applied foreign proteins are taken up by and processed in MHC class II pathway in antigen present cells and subsequently activate humoral immune response (Heikema A, et al. Generation of heat shock protein-based vaccines by intracellular loading of gp96 with antigenic peptides. Immunol Lett 1997 June 1;57(1-3): 69-74), but can not induce effectively the development of tumor-specific CTL, and therefore can not be tumor preventive and therapeutic. So, conferring exogenously applied MUC1 with activities of specific CTL generating is crucial for the development of the most effective MUC1-based recombinant protein that will induce preventive and therapeutic CTLs to human MUC1 expressing carcinomas including breast cancer, ovarian cancer, colon cancer, prostate cancer and lung cancer.

Several experiments have demonstrated that heat-shock proteins (HSP) can confer an antigenic peptide with CTL generating properties and thereby activate human tumor-specific CTL to kill tumor cells in an effective way. Udono H reported in 1993 and 1994 that immunization of mice with HSP complex derived from autologous cancer can elicit cancer-specific immunity and that the specificity of this immune response is caused by tumor-derived peptides bound to the heat shock proteins (Udono H and P. K. Srivastava.1993.J. Exp. Med.178:1391-1396) (Udono H and P. K. Srivastava. 1994. J. Immunol.152:5398-5403).

Arnold demonstrated that a single immunization with the heat shock protein preparations isolated from P815 cells (of DBA/2 origin) induces cytotoxic T lymphocytes (CTLs) in mice with identical major histocompatibility complex (MHC, H-2d) as well as in mice with a different MHC (H-2b), indicating that HSP induced specificity are not limited to the MHC class I (Arnold D, Faath S, Rammensee H, Schild H. Cross-priming of minor histocompatibility antigen-specific cytotoxic T cell upon immunization with the heat shock protein gp96. J. Exp Med1995 September 1;182(3):885-9).

In fact, it has been demonstrated that a HSP 65 T cell epitope fusion protein can induce CTLs in mice which recognize the T cell epitope comprising 8-10 amino acid residues in fusion protein, which indicates that HSP can help the its fused partner into MHC I pathway and induce specific CTL against T cell epitope. It has also been demonstrated that HSP—tumor antigen fusion protein can be taken up and processed by dendritic cells and directly elicit dendritic cells to express a high level of costimulatory molecule-B7 molecule. Processed tumor antigen binds with MHC class I molecules and is expressed on the surface of dendritic cells, is presented to a specific CTL, stimulates a CTL, elicited a CTL to kill the tumor cell which has expressed tumor antigen (A proposed mechanism for the induction of cytotoxic T lymphocyte production by heat shock fusion proteins, Immunity, 2000, 12, 1-20). In vivo, CTL induced by APCs, especially dendritic cells which express B7 molecules and MHC class I molecules-tumor antigen peptides, can kill tumor cells expressing tumor antigen. This killing needs no recognition of costimulatory molecules including B-7 on tumor cell surface.

Since HSP65 can be combined with a peptide to elicit a CTL immune response, the combination of HSP65 with a peptide analogous to MUC1 will elicit an immune response, both in vitro and in vivo. An HSP—tumor antigen fusion protein that contained MUC1 peptide(s) would be preventive and therapeutic for carcinomas, including human carcinomas.

Embodiments of the invention are thus provided having a recombinant fusion protein comprising Bacillus Calmette Guérin heat shock protein 65 and, e. g. , 1 to 5 copies of the epitope of MUC1. The recombinant fusion proteins may contain one, two, three, four or five copies, preferably two copies, of the epitope of MUC1. The epitope of MUC1 means a peptide fragment of MUC1 that can generate MUC1 specific cytotoxic T lymphocytes (CTL) and can be recognized by T cell receptor (TCR) of the CTL. In the recombinant fusion protein, the Bacillus Calmette Guérin heat shock protein 65 is preferably located at the amino terminal of the fusion protein and the epitope of MUC1 is located at the carboxy terminal of the fusion protein. In a preferred embodiment the epitope of MUC1 has the amino acid sequence shown in SEQ ID NO: 4. Alternatively, the epitope of MUC1 has at least a portion of the amino acid sequence shown in SEQ ID NO: 4, or at least a portion of a sequence having 80%, 85%, 90%, or 95% identity to SEQ ID NO: 4. In another preferred embodiment the recombinant fission protein has the amino acid sequence shown in SEQ ID NO: 2. Alternatively, the recombinant fusion protein has at least a portion of the amino acid sequence shown in SEQ ID NO: 2, or at least a portion of a sequence having 80%, 85%, 90%, or 95% identity to SEQ ID NO. 2. Portion lengths may be, for example, at least 3 amino acids, at least 6, at least 8, less than 100, less than 50, less than 20, less than 15 amino acids, or any combination thereof. Examples of DNA or RNA sequences suitable for generating MUC1 epitopes are shown in SEQ ID NO: 3.

An embodiment described herein is a recombinant fusion protein which is preventive and/or therapeutic to human MUC1 expressing carcinoma (breast cancer, ovarian cancer, colon cancer, prostate cancer and lung cancer).

An embodiment described herein is a nucleic acid molecule encoding a recombinant fusion protein as described herein.

An embodiment described herein is a plasmid containing a nucleic acid as described herein.

An embodiment described herein is a host cell transformed with a plasmid as described herein.

An embodiment described herein is a method for preparing a recombinant fusion protein.

An embodiment described herein is a vaccine formulation, comprising a recombinant fusion protein as described herein.

An embodiment described herein is a recombinant fusion protein as described herein.

An embodiment described herein is a nucleic acid molecule encoding a recombinant fusion protein as described herein. In a preferred embodiment, a nucleic acid molecule includes the nucleic acid sequence shown in SEQ ID NO: 1. Alternatively, a nucleic acid having at least a portion of a sequence as shown in SEQ ID NO: 1 may be used, or a sequence having at least a portion that has 80%, 85%, 90%, or 95% identity to SEQ ID NO: 1 may be used.

An embodiment described herein is a method for preparing a recombinant fusion protein as described herein, comprising the step of culturing the aforesaid transformed host cell.

An embodiment described herein is a vaccine formulation, comprising the recombinant fusion protein Bacillus Calmette Guérin heat shock protein 65 and 1 to 5 copies of the epitope of MUC1 as the active ingredient and a biologically acceptable excipient.

An embodiment described herein is a method for use of a recombinant fusion protein Bacillus Calmette Guérin heat shock protein 65 and 1 to 5 copies of the epitope of MUC1 in the preparation of a pharmaceutical preparation for preventing and/or treating MUC-1 expressing carcinomas.

Recombinant HSP65-MUC1-ME fusion proteins described herein are preventive and/or therapeutic to human MUC1 expressing carcinoma (breast cancer, ovarian cancer, colon cancer, prostate cancer and lung cancer). The recombinant anti-tumor fusion proteins consisting of Bacillus Calmette Guérin (BCG) heat shock protein 65 (hsp65) and the epitope of MUC1(MUC1ME) can be produced in E. coli. by fermentation. When administered to a human subject, HSP65 leads MUC-1ME to dendritic cells where hsp65 assists MUC1ME to be processed in MHC class I pathway. Processed MUC1 peptides are expressed, associated with MHC class I molecules, on dendritic cells. In addition, HSP65 stimulates dendritic cells to express co-stimulatory molecules efficient to provide second activating signals to cytotoxic T lymphocytes (CTL). The dendritic cells consequently cross-activate MUC1 specific CTL which are potent MUC1 expressing tumor cell killers.

Other uses for fusion proteins described herein include uses as research tools, research reagents, and as agents for the creation of antibodies that may be used therapeutically, diagnostically, or as research reagents and tools. For example, the fusion proteins may be packaged, preferably with biologically acceptable agents and/or excipients, for use with in vitro in cell or tissue culture protocols to study aspects of immune system mechanisms. Antibodies to the fusion proteins are useful for identifying the patterns of localization of the fusion proteins in vitro or in vivo, and in cellular or tissue samples.

The identity of a protein or nucleic acid sequence is frequently established based on a sequence alignment of the DNA, RNA, or amino acids.

Multiple alignments of such sequences are important tools in studying biomolecules. The basic information they provide is identification of conserved sequence regions. This is very useful in designing experiments to test and modify the function of specific proteins, in predicting the function and structure of proteins, and in identifying new members of protein families. Sequences can be aligned across their entire length (global alignment) or only in certain regions (local alignment). This is true for pairwise and multiple alignments. Global alignments with respect to polynucleic acids or polypeptides usually require gaps (representing insertions/deletions) while local alignments can usually avoid gaps by aligning regions between gaps. In a sequence alignment, letters arranged over one another are called matched. If two matched letters are equal, the match is called an identity otherwise the match is called a substitution or mismatch. An insertion or deletion is one or more letters aligned against a gap (-) and is considered the same as a mismatch for percent identity purposes.

In some cases a determination of the percent identity of a peptide to a sequence set forth herein may be required. In such cases, the percent identity is measured in terms of the number of residues of the peptide, or a portion of the peptide. Thus a peptide of 10 residues would be 90% identical to SEQ ID NO 1 if nine of the residues of the peptide were determined to be matched to SEQ ID NO 1. A peptide or polypeptide of, e. g. , 90% identity, may also be a portion of a larger peptide; for example, a peptide of 100 residues that has a portion that is 10 residues in length that is matched to 9 residues of SEQ ID NO 2 would have 90% identity with SEQ ID NO 2.

The amino acid residues described herein employ either the single letter amino acid designator or the three-letter abbreviation. Abbreviations used herein are in keeping with the standard polypeptide nomenclature, J. Biol. Chem., (1969), 243, 3552-3559. All amino acid residue sequences are represented herein by formulae with left and right orientation in the conventional direction of amino-terminus to carboxy-terminus.

### BRIEF DESCRIPTION OF THE DRAWINGS

FIG. 1 shows the identification of HSP65-MUC1-ME producing bacterial clone by electrophoresis. Lane A: Digested pET28a-plasmid carrying HSP65-MUC1-ME gene, the fragment of HSP65-MUC1-ME gene was indicated by an arrow; Lane M: DL 2,000 markers, 2000 bp, 1000 bp, 750 bp.

FIG. 2 shows identification of recombinant HSP65-MUC1-ME by SDS-PAGE.

A: SDS-PAGE. M. protein molecular weight marker (96KD 66KD 43KD 36KD 20.1KD 14.4KD); a, b: purified recombinant HSP65-MUC1-ME

B: Western blot identification. a': band in a lane showed in A hybridized with HSP65 monoclonal antibody; b': band in b lane showed in A hybridized with MUC1 monoclonal antibody FIG. 3 shows 8R-MUC1-3C fusion protein.

A: Agarose gel electrophoresis of 8R-MUC1-3C fusion protein encoding DNA. a: 8R-MUC1-3C fusion protein encoding DNA released from pET28a plasmid with NcoI and XhoI, indicated by an arrow; m: DNA molecular weight marker.

B: SDS-PAGE analysis of 8R-MUC1-3C fusion protein. a, b: 8R-MUC1-3C fusion protein purified from E. coli, indicated by an arrow; m: protein molecular markers.

C. Amino acid sequence of 8R-MUC1-3C fusion protein.

FIG. 4 shows the activity of MUC1 specific CTL generated in mice immunized with HSP65-MUC1-ME.

FIG. 5 shows the growth inhibition of B 16 cells pulsed with MUC13C in mice immunized with HSP65-MUC1-ME. A: mice injected with PBS, B 16 cells are in black color; B: mice injected with HSP65-MUC1-ME; C: Average weight of B 16 cells in group A (mice injected with PBS) and group B (mice injected with HSP65-MUC1-ME).

FIG. 6 shows the effects of immunization with HSP65-MUC1-ME on the survival of tumor bearing mice.

FIG. 7 shows the growth inhibition of B 16 cells transfected with MUC13C gene in mice immunized with HSP65-MUC1-ME.

FIG. 8 shows the activity of CTLs generated by in vitro immunization of DCs loaded with HSP65-MUC1-ME.

### EXAMPLES

### Example 1

Construction of BCG heat shock protein 65 HSP 65 and the epitope of MUC1 (MUC1 ME) fusion gene

BCG was provided by Chang Chun Institute of Biological Products, China. BCG genome was extracted according to the methods as described in Molecular Cloning (J. Sambrook. Isolation of high-molecule-weight DNA from mammalian cells, 9.16-9.22, Cold Spring Harbor Laboratory Press, Molecular Cloning, 1989).

Taking newly extracted BCG genome DNA as template, HSP-65 encoding genes was amplified by PCR using synthetic primers. The forward primer is 5′ CCATGGCCAAG ACAATTGCG 3′ (SEQ ID NO:5), which contains a NcoI site. The reverse primer is 5′ CGAATTCGCTAGCCATATGGAA ATCCATGCCACCCAT 3′ (SEQ ID NO:6), which contains an EcoRI site.

PCR was performed by adding the following reagents in a 500 l Eppendorf tube: template cDNA 5 l (mmol/L), 10×PCR buffer 5 l, dNTPs(10 mmol/L) 1 l, the forward and reverse primer (0.01 mmol/L) 0.5 l each, Taq DNA polymerase(5u/l) 0.25 l. The volume of the reaction mixture was adjusted to 50 l with deionized H₂O. The mixture was agitated, then covered with a drop of mineral oil. The reaction was performed according to following procedure: 94, 30 seconds; 55, 1 minute; 72, 2 minutes. After the reaction was allowed to run for 30 cycles, the mixture was extended for 10 minutes at 72.

PCR products were electrophoresed in 1% agarose gel and a DNA fragment about 1638 bp was observed. DNA was recovered from the gel and used as templates to synthesize BCG heat shock protein 65 HSP 65 and the epitope of MUC1 (MUC1ME) fusion gene by PCR. The forward primer is: 5′ ttc gcc atg gcc aag aca att gcg 3′ (SEQ ID NO:7) and the reverse primer is: 5′ ggc cgc aag ctt tta tca cag agc cgg acg gtt gtc cgg agc aga ggt aac acc gtg agc cgg cgg agc ggt aga acc cgg agc cgg acg ggt gtc cgg agc aga ggt aac acc gtg agc cgg cgg agc ggt aga acc gaa ttc gct agc cat atg caa atc 3′ (SEQ ID NO:8). Reaction conditions: 94° C., 30″; 55° C., 1′; 72° C., 4′; 30 cycles; 72° C., 10′.

The PCR product was digested with NcoI and HindIII, purified and ligated to pET28a plasmids (Novagen, America ). The plasmids were transformed into BL21 DE3 bacteria (Novagen, America) and the insert in the plasmid was identified to be the encoding gene (SEQ ID NO: 1) of BCG heat shock protein 65 HSP 65 and the epitope of MUC1 (MUC1ME) fusion protein by DNA sequencing. This fusion gene encodes BCG heat shock protein 65 HSP 65 and the epitope of MUC1 (MUC1ME) fusion protein (HSP65-MUC1-ME) with the amino acid sequence indicated in SEQ ID NO: 2.

The bacterial clone producing HSP65-MUC1-ME was obtained by the following procedures:

Firstly, competent bacteria were prepared. The BL21 DE3 bacteria were streaked onto the surface of an agar plate by using an inoculating loop. The inoculated plate was incubated for 12-16 hours at 37° C. A single bacterium colony was picked into 2 ml of LB medium in a 15 ml tube and the tube was agitated vigorously for 1 minute. 1 ml of the bacteria suspension was transferred into 100 ml LB in a 1-liter flask, followed by incubation at 37° C. with vigorous agitation (225 r/min) until the medium OD₆₀₀ reaches 0.5 (within about three hours). The cultured bacteria were cooled on ice for 2 hours and collected by centrifugation at 2,500 g at 4 for 20 minutes. The bacterial pellet was resuspended in 100 ml ice-cold Trituration buffer (100 mmol/L CaCl₂, 70 mmol/L MgCl₂, 40 mmol/L Acetate Acid, pH 5.5). The bacteria were incubated on ice for 45 minutes, followed by centrifugation at 1,800 g at 4 for 10 minutes. The bacterial pellet was resuspended in 10 ml ice cold Trituration buffer and a 200 ul aliquot of the bacterium suspension with 15% glycerol was dispensed into a sterile Eppendorf tube. The bacteria in the Eppendorf tube were competent cells for transformation of plasmid and were stored at −70.

Secondly, pET28a plasmids carrying HSP65-MUC1-ME encoding genes were transformed into BL21 DE3 bacteria. A 200 μl aliquot of competent cells in a Eppendorf tube was thawed on ice. 3 μl DMSO and 0.5 g pET28a plasmids carrying HSP65-MUC1-ME encoding genes were added and mixed gently. The tube was incubated on ice for 30 minutes and on a rack in a preheated 42 circulating water baths for 45 seconds. The bacteria in the tube were allowed to cool on ice for 1-2 minutes and then transferred into a flask containing 2 ml LB medium, followed by incubation at 37 with rotation at 225 r/min for 1 hour. The bacteria were collected by centrifugation at 4,000×g for 5 minutes and were resuspended in 200 μl LB medium. An appropriate volume of transformed competent cells was spread onto an agar plate containing Kanamycin (50 μg/ml). The plate was inverted and incubated at 37° C. for 12-16 hours.

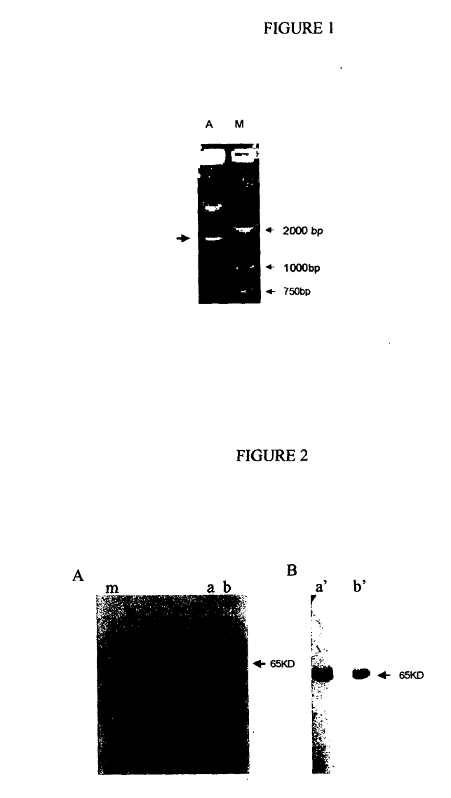

Thirdly, the HSP65-MUC1-ME producing bacterium clone was collected, identified by restriction enzyme digestion-agarose gel electrophoresis (FIG. 1) and DNA sequencing (SEQ ID NO:1), freeze-dried and stored at −20° C.

### Example 2

Production and purification of HSP65-MUC1-ME fusion protein

HSP65-MUC1-ME fusion protein was produced with IPTG as inducers in 10-liter fermentor. The bacteria harvested were lysed by using a homogenizer and HSP65-MUC1-ME fusion proteins released were purified by hydrophobic chromatography, ion-exchange chromatography successively. The concentration of HSP65-MUC1-ME fusion protein was determined by the Micro BCA protein assay reagent kit. After being sterilized through 0.2 μM membrane filtration, the purified recombinant HSP65-MUC1-ME was freeze-dried and stored at −70° C.

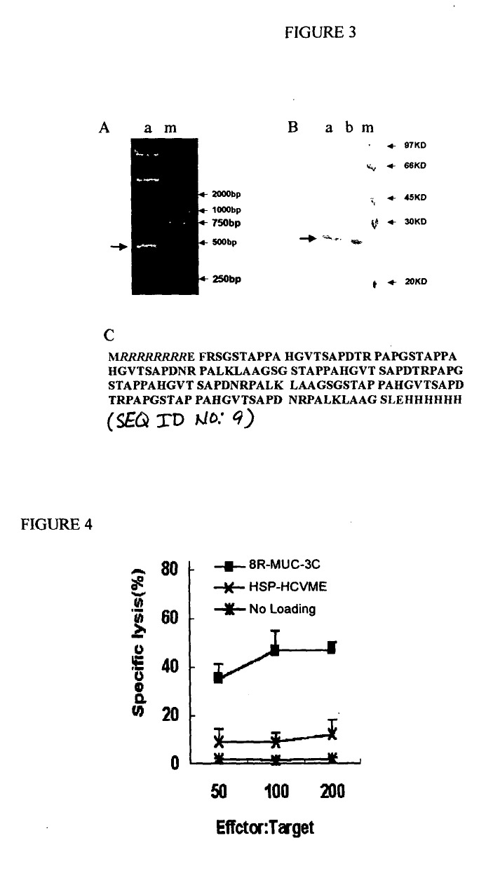

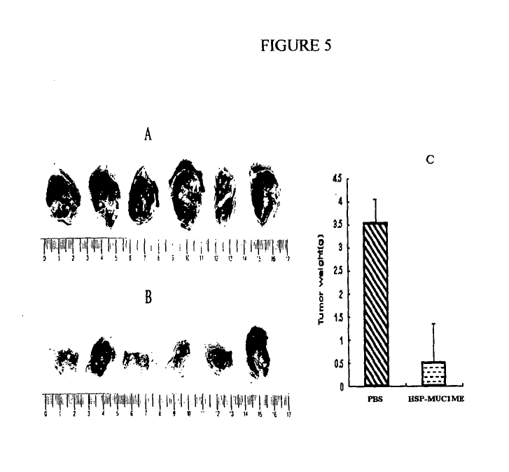

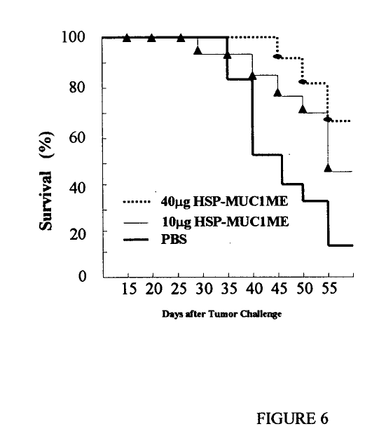

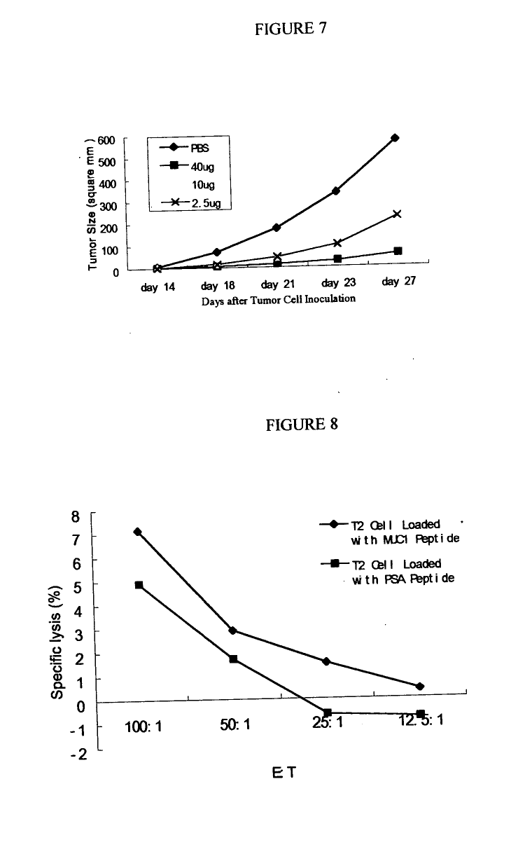

The purity and identity of the fusion protein was analyzed by 12% SDS-PAGE gels (FIG. 2) stained with Coomassie blue (Bio-Rad) and by a Western blot using HSP-65 monoclonal antibodies (MBL, Japan) and MUC1 monoclonal antibodies (BD Pharmingen) respectively (FIG. 2).

### Example 3

Induction of MUC1-specific tumor cytotoxic T lymphocytes by HSP65-MUC1-ME in mice

C57BL/6 mice were inoculated with 10 μg of HSP65-MUC1-ME fusion protein in 200 1 PBS in the legs subcutaneously on day 0, 14, 28. On day 5 after the last inoculation, the mice were sacrificed and spleen cells were isolated, cultured for 5 days in the presence of HSP65-MUC1-ME (10 μg/ml) and used as effector cells. The B16 cells pulsed with 8R-MUC1-3C (FIG. 3) or HSP-HCV recombinant protein respectively, and B 16 without pulsing were used as target cells. Splenocytes within groups were pooled. Serial dilutions of resultant spleen cells were cultured with 1×10⁴ target cells labeled with ⁵¹Cr in the wells of 96-well round bottom plate. After 6 hours incubation at 37° C., the plate was centrifuged at 3000 rpm for 5 minutes and then 100 μl supernatant was collected for gamma radiation counting. The percent specific release was calculated ((specific release-spontaneous release)/(total release-spontaneous release))×100. The spontaneous release was less than 15%. The results showed that MUC1 specific CTLs were generated in mice immunized with HSP65-MUC1-ME.

The result showed that MUC1 specific CTL were generated in mice immunized with HSP65-MUC1-ME (FIG. 4).

### Example 4

Growth inhibition of B16 cells pulsed with 8R-MUC1-3C in mice by using HSP65-MUC1-ME

C57BL/6 mice were inoculated with 10 g of HSP65-MUC1-ME in 200 μl PBS on day 0, 14, 28; PBS group was set up as control. On day 5 after the last inoculation, B16 cells pulsed with 8R-MUC1-3C fusion protein (FIG. 3) or B16 without pulsing were administered to the mice in the legs subcutaneously. On day 15 after tumor inoculation, the mice were sacrificed and the tumor was isolated and weighed. The results demonstrate that the tumor growth was inhibited to variable extents in C57BL/6 mice immunized with recombinant HSP65-MUC1-ME (FIG. 5).

### Example 5

The life span prolongation of mice bearing B16 cells pulsed with 8R-MUC1-3C fusion protein by using HSP65-MUC1-ME

C57BL/6 mice were inoculated with 10 μg of recombinant HSP65-MUC1-ME fusion protein in 200 μl PBS on day 0, 14, 28. The control mice were injected with PBS. On day 5 after the last inoculation, B16 cells pulsed with 8R-MUC1-3C fusion protein or B16 cells without pulsing were administered to the mice in the legs subcutaneously. The injection area was observed and palpated for the presence of a tumor nodule. The results showed that the life span was prolonged in mice immunized with recombinant HSP65-MUC1-ME (FIG. 6).

### Example 6

Growth retardation of B16 tumor cells transfected with MUC13C peptide encoding gene in mice by HSP65-MUC1-ME

On day 0, 14 and 28, C57BL/6 female 12 weeks old mice were immunized with 10 μg HSP65-MUC1-ME in 200 μl PBS in the legs subcutaneously. On day 5 after the last inoculation, 1×10⁵ B16 cells transfected with VR1055 plasmids carrying MUC13C (FIG. 3) encoding gene lacking of 8R genetic codons were administered to the mice in the back subcutaneously. On day 14, the tumor nodules began to be palpable and be measured daily. The results showed that the growth of tumor cells transfected with MUC13C (FIG. 3) encoding gene was inhibited in mice immunized with recombinant HSP65-MUC1-ME.

### Example 7

Induction of human CTLs by in vitro immunization with autologous dendritic cells pulsed with HSP65-MUC1-ME

Fresh blood from healthy donor HLA-A⁺₂ individuals was diluted with PBS/EDTA and added into 50 ml tube containing 12.5 ml Ficoll-hypaque with caution to avoid disturbing the Ficoll-hypaque. The tube was centrifuged at 3000 rpm for 20-25 minutes at RT without brake. The upper supernatant was discarded and the interface cells were collected and transferred into a 50 ml fresh tube. PBS/EDTA buffer was added into the tube containing the collected cells up to 45 ml. By centrifugation, the cells were washed at 1800 rpm at 4° C. for 10 minutes once, washed at 1200 rpm at 4° C. for 7 minutes and washed with PBS/EDTA/human serum buffer, without brake, at 1200 rpm at 4° C. for 7 minutes. The resultant cells were counted and adjusted to a concentration of 5×10⁶ cells/ml with PBS/EDTA/human serum buffer (cold). 3 ml of the cell suspension was added onto the surface of 6 ml of 52% Percoll in a 10 ml tube with caution. The tube was centrifuged at 2000 rpm at 4° C. for 20 minutes without brake. The interface cells were collected into a fresh tube. The collected cells were washed twice with cold PBS/EDTA/human serum buffer at 1300 rpm at 4° C. for 10 minutes without brake. After being washed, the cells, among which 50% were monocytes, were filtered through a nylon mesh and counted.

The cells rich in monocytes were cultured in 2 ml complete medium with final concentration of 1×10⁶ cells/ml in a well of a 12-well plate in an atmosphere of 5% CO₂ at 37° C. for 2 hours. After removal of non-adherent cells, the adherent cells were incubated in 2 ml of complete IMDM medium supplemented with 100 ng (200 U) /ml GM-CSF and 200 U/ml IL-4 in an atmosphere of 5% CO₂ at 37 for 5 days. On day 3, the medium was refreshed once. On day 5, the cells, already differentiated into immature dendritic cells, were incubated with HSP65-MUC1-ME 100 μg/ml for 2 more days. On the day 7, the cells (mature dendritic cells) were harvested and analyzed for CD40, CD19, CD80, CD83, CD56, HLA-DR, HLA-A-2 expressions by staining with specific monoclonal antibodies, followed by FAS assay.

20×10⁶ of PBMC were incubated in 1 ml of PBS/2 mM EDTA/5% human serum buffer supplemented by 1:1000 dilution of CD14, CD56, CD19, CD4, γδTCR monoclonal antibodies for 30 minutes on ice with shaking, followed by removing antibody coated cells using anti-mouse IgG magnetic beads. The CD45⁺ CD4⁻ cells (CD8⁺ T cells) were sorted to 99.9 purity and subjected to three DC in vitro immunization at 7 days interval.

The immunized CD8⁺ T cells were seeded into 96 well round bottom plate incubated with ⁵¹Cr labeled T2 cells pulsed with peptide (APDTRPAP, STAPPAHGV, 10-100 μg) at 37° C., 5% CO₂ for 4 h. After centrifugation for 5 minutes, half of supernatant were collected and assayed in a gamma counter for ⁵¹Cr release. The percent specific release was calculated ((specific release-spontaneous release)/(total release-spontaneous release))×100. The spontaneous release was less than 15%.

The results show that mature human autologous DCs pulsed with recombinant HSP65-MUC1-ME fusion protein can induce MUC1-specific CTL which kill HLA-A2 tumor cells loaded with MUC1 peptides.

1. A recombinant fusion protein comprising Bacillus Calmette Guérin heat shock protein 65 and 1 to 5 copies of an epitope of MUC1.

2. A method for preparing a recombinant fusion protein comprising culturing a host cell transformed with a plasmid that comprises a nucleic acid molecule encoding the recombinant fusion protein of claim 1.

3. A composition comprising the recombinant fusion protein of claim 1 as the active ingredient and a biologically acceptable excipient.

4. A method of making a medicament, comprising preparing a pharmaceutical preparation comprising the recombinant fusion protein of claim 1 and a biologically acceptable excipient.

5. The recombinant fusion protein of claim 1, wherein the Bacillus Calmette Guérin heat shock protein 65 is located at the amino terminal portion of the fusion protein and the epitope of MUC1 is located at the carboxy terminal portion of the fusion protein.

6. A nucleic acid molecule encoding the recombinant fusion protein of claim 5.

7. A method for preparing a recombinant fusion protein, comprising culturing a host cell transformed with a plasmid comprising a nucleic acid molecule encoding the recombinant fusion protein of claim 5.

8. A composition comprising the recombinant fusion protein of claim 5 as the active ingredient and a biologically acceptable excipient.

9. A method of making a medicament, comprising preparing a pharmaceutical preparation comprising the recombinant fusion protein of claim 5 and a biologically acceptable excipient

10. The recombinant fusion protein according to claim 1, wherein at least one epitope of MUC1 comprises an amino acid sequence as shown in SEQ ID NO: 4.

11. A nucleic acid molecule encoding the recombinant fusion protein of claim 10.

12. A method for preparing a recombinant fusion protein, comprising culturing a host cell transformed with a plasmid comprising a nucleic acid molecule encoding the recombinant fusion protein of claim 10.

13. A composition comprising the recombinant fusion protein of claim 10 as the active ingredient and a biologically acceptable excipient.

14. A method of making a medicament, comprising preparing a pharmaceutical preparation comprising the recombinant fusion protein of claim 10 and a biologically acceptable excipient.

15. The recombinant fusion protein according to claim 1, wherein at least one epitope of MUC1 comprises an amino acid sequence having a length of at least eight amino acids and having at least 85% identity to SEQ ID NO: 4.

16. A nucleic acid molecule encoding the recombinant fusion protein of claim 15.

17. A method for preparing a recombinant fusion protein, comprising culturing a host cell transformed with a plasmid comprising a nucleic acid molecule encoding the recombinant fusion protein of claim 15.

18. A composition comprising the recombinant fusion protein of claim 15 as the active ingredient and a biologically acceptable excipient.

19. A method of making a medicament, comprising preparing a pharmaceutical preparation comprising the recombinant fusion protein of claim 15 and a biologically acceptable excipient.

20. The recombinant fusion protein according to claim 1, comprising an amino acid sequence as shown in SEQ ID NO: 2.

21. A nucleic acid molecule encoding the recombinant fusion protein of claim 20.

22. A method for preparing a recombinant fusion protein, comprising culturing a host cell transformed with a plasmid comprising a nucleic acid molecule encoding the recombinant fusion protein of claim 20.

23. A composition comprising the recombinant fusion protein of claim 20 as the active ingredient and a biologically acceptable excipient.

24. A method of making a medicament, comprising preparing a pharmaceutical preparation comprising the recombinant fusion protein of claim 20 and a biologically acceptable excipient.

25. The recombinant fusion protein according to claim 1, comprising an amino acid sequence having at least 85% identity to SEQ ID NO: 2.

26. A nucleic acid molecule encoding the recombinant fusion protein of claim 25.

27. A method for preparing a recombinant fusion protein, comprising culturing a host cell transformed with a plasmid comprising a nucleic acid molecule encoding the recombinant fusion protein of claim 25.

28. A composition comprising the recombinant fusion protein of claim 25 as the active ingredient and a biologically acceptable excipient.

29. A method of making a medicament, comprising preparing a pharmaceutical preparation comprising the recombinant fusion protein of claim 25 and a biologically acceptable excipient.

30. A nucleic acid molecule encoding the recombinant fusion protein of claim 1.

31. The nucleic acid molecule of claim 30, comprising a nucleic acid sequence as shown in SEQ ID NO:1.

32. A plasmid comprising the nucleic acid of claim 31.

33. A host cell transformed with the plasmid as claimed in claim 32.

34. The nucleic acid molecule of claim 30, comprising a nucleic acid sequence having at least 85 % identity to SEQ ED NO:1.

35. A plasmid comprising the nucleic acid of claim 34.

36. A host cell transformed with the plasmid as claimed in claim 35.

37. A plasmid comprising the nucleic acid of claim 30.

38. A host cell transformed with the plasmid as claimed in claim 37.
